(19) United States
(12) Patent Application Publication
(10) Pub. No.: US 2020/0149036 A1
(43) Pub. Date: May 14, 2020

(54) METHODS TO DISTINGUISH RNA AND DNA IN A COMBINED PREPARATION

(71) Applicant: Exosome Diagnostics, Inc., Waltham, MA (US)

(72) Inventors: Daniel ENDERLE, Martinsried (DE); Johan SKOG, Lincoln, MA (US); Phillipp TORKLER, Munich (DE); Alexandra SPIEL, Munchen (DE); Mikkel NOERHOLM, Martinsried (DE)

(21) Appl. No.: 16/473,604

(22) PCT Filed: Jan. 2, 2018
(86) PCT No.: PCT/US2018/012099

§ 371 Date: Jun. 25, 2019

Related U.S. Application Data
(60) Provisional application No. 62/441,529, filed on Jan. 2, 2017.

(51) Int. Cl. C12N 15/10; C12Q 1/6806; C12Q 1/6874

(52) U.S. Cl. CPC C12N 15/1093; C12Q 1/6874; C12Q 1/6806

(57) ABSTRACT

The invention provides a series of steps to distinguish RNA from DNA in assays downstream of a combined isolation. This enables a wide diversity of RNAs and/or DNAs, to be efficiently detected.

### RELATED APPLICATION

This application claims the benefit of U.S. Provisional Application No. 62/441,529, filed Jan. 2, 2017, the contents of which are incorporated herein by reference in their entirety.

### FIELD OF THE INVENTION

The present invention is in the technical field of biotechnology. More particularly, the present invention is in the technical field of molecular biology. In molecular biology, molecules, such as nucleic acids, can be isolated from human sample material, such as tissue and various biofluids, and further analyzed with a wide range of methodology.

### BACKGROUND

Nucleic acids extracted from, e.g., plasma or serum for use in liquid biopsies have a generally low nucleic acid content, which necessitates a need for maximizing extraction efficiency. Only recently has it been understood that some sample types, such as plasma or serum, contain RNA and DNA that is of high value, low amount, and difference in biological source, creating the need for assays that distinguish between these nucleic acids.

Moreover, DNA and RNA are released into circulation by two fundamentally different biological processes. While the majority of DNA in plasma is from apoptotic or necrotic cells, the majority of RNA is generated by living and actively dividing cells. Distinguishing between the origin or source of components within nucleic acid samples is desirable.

Some extraction methods to isolate nucleic acids from a given sample specifically isolate only the RNA (ribonucleic acid) or only the DNA (deoxyribonucleic acid). However, some methods aim at isolating both nucleic acids together, increasing extraction efficiency but relying on downstream assay technology to distinguish between both types of molecules.

Accordingly, there is a need for reliable identification of nucleic acids within a mixture. The present disclosure is directed to these, and other, important ends.

### SUMMARY OF THE INVENTION

The present invention is directed to methods for identifying DNA and RNA in an isolate or a mixture comprising both, by specifically modifying and detecting either the RNA component or the DNA component. In some embodiments, the RNA component is modified and subjected to further analyses, while the DNA component remains unmodified. In alternative embodiments, the DNA component is modified and subjected to further analyses, while the RNA component remains unmodified.

In some embodiments, the disclosure provides a method for identifying a nucleic acid component in a mixture comprising: (a) providing a mixture comprising at least an RNA component and a DNA component; (b) selectively or specifically modifying the RNA component to provide a modified RNA component and an unmodified DNA component, or specifically modifying the DNA component to provide a modified DNA component and an unmodified RNA component; and (c) performing a molecular assay which identifies the modified RNA component or the modified DNA component.

In some embodiments, (a) comprises a mixture of exosomal RNA, exosomal DNA and cell-free DNA (cfDNA), a mixture of exosomal RNA and exosomal DNA, or even a mixture of exosomal RNA and cfDNA. In some embodiments, (a) comprises a mixture of RNA and DNA from fresh, frozen or formalin fixed paraffin embedded (FFPE) tissue.

In some embodiments, (b) comprises chemically modifying the nucleic acid bases or backbone of the RNA component.

In some embodiments, (b) comprises modifying the nucleic acid sequence of the RNA component.

In some embodiments, (b) comprises adding a nucleic acid sequence to the RNA component.

In some embodiments, (b) comprises contacting the RNA component with one or more enzymes.

In some embodiments, (b) comprises contacting the RNA component with one or more reverse transcription enzymes.

In some embodiments, (b) comprises tagging the RNA component with a nucleic acid sequence.

In some embodiments, (b) comprises performing a first strand cDNA synthesis and contacting the RNA component with a reverse transcription enzyme and one or more tagged primers as a substrate for reverse transcription, thereby tagging the RNA component with a nucleic acid sequence. For example, the one or more tagged primers comprise an oligonucleotide that comprises random nucleotides and nonrandom nucleotides, or even comprise tagged random hexamers.

In some embodiments the nonrandom nucleotide portion of the oligonucleotide comprises a G-C content in the range of about 40-60%, such as about 40-50%, or even about 40-45%; the nonrandom nucleotide portion of the oligonucleotide comprises a nucleic acid sequence that is of size the range of about 1-40, 1-20, or even 1-5 nucleotides; and/or the nonrandom nucleotide portion of the oligonucleotide comprises a nucleic acid sequence that is unique to the genome of interest.

In some embodiments, the random nucleotide portion of the oligonucleotide is depleted of nucleotide combinations priming at highly abundant RNAs.

In some embodiments, (b) comprises modifying at least 50%, such as at least 60%, at least 70%, at least 80%, at least 85%, at least 90%, at least 95%, at least 98%, or even 100% of the RNA component in the mixture.

In some embodiments, (c) comprises detecting the modified RNA component by a polymerase chain reaction (PCR)-based assay.

In some embodiments, (c) comprises detecting the modified RNA component by next generation sequencing (NGS).

In some embodiments, (c) comprises detecting the modified RNA component and the DNA component by NGS in a single combined library.

In some embodiments, (c) comprises performing NGS under conditions sufficient to allow for efficient capture of both cDNA derived from the RNA component and the DNA component in a single combined library.

In some embodiments, (c) comprises distinguishing sequence reads from RNA and DNA in a NGS dataset.

In some embodiments, (c) comprises identifying the genomic strand of RNA origin.

In some embodiments, (b) comprises chemically modifying the nucleic acid bases or backbone of the DNA component

In some embodiments, (b) comprises modifying the nucleic acid sequence of the DNA component.

In some embodiments, (b) comprises adding a nucleic acid sequence to the DNA component.

In some embodiments, (b) comprises contacting the DNA component with one or more enzymes.

In some embodiments, (b) comprises tagging the DNA component with a nucleic acid sequence.

In some embodiments, (b) comprises tagging the DNA component with an oligonucleotide that comprises random nucleotides and nonrandom nucleotides.

In some embodiments, the nonrandom nucleotide portion of the oligonucleotide comprises a nucleic acid sequence that is unique to the genome of interest.

In some embodiments, (b) comprises modifying at least 50%, such as at least 60%, at least 70%, at least 80%, at least 85%, at least 90%, at least 95%, at least 98%, or even 100% of the DNA component in the mixture.

In some embodiments, (c) comprises detecting the modified DNA component by a polymerase chain reaction (PCR)-based assay.

In some embodiments, (c) comprises detecting the modified DNA component by next generation sequencing (NGS).

In some embodiments, (c) comprises detecting the modified DNA component and the RNA component by NGS in a single combined library.

In some embodiments, (c) comprises identifying the genomic strand of DNA origin.

In some embodiments, the method further comprises performing a transcriptome analysis and detecting genomic or epigenomic features of the genome.

In some embodiments, the method further comprises determining relative abundance of the RNA component and the DNA component in order to identify copy number variations present in the mixture.

In some embodiments, the method further comprises predicting expressed, relevant tumor Neoantigens.

In some embodiments, the method further comprises simultaneous mutation detection in the RNA component and the DNA component of the mixture.

Various aspects and embodiments of the invention will now be described in detail. It will be appreciated that modification of the details may be made without departing from the scope of the invention. Further, unless otherwise required by context, singular terms shall include pluralities and plural terms shall include the singular.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representations as to the contents of these documents are based on the information available to the applicants and do not constitute any admission as to the correctness of the dates or contents of these documents.

### BRIEF DESCRIPTION OF THE FIGURES

FIG. 1 is a schematic drawing of an exemplary process to distinguish RNA and DNA in a combined isolation using tagged random hexamers followed by ligation-based NGS. The two different types cluster sequences denote either RNA or DNA.

FIG. 2 provides an exemplary experimental outline for identifying components of pooled nucleic acids from four separate isolations.

FIG. 3 shows a spike-in recovery demonstrating successful RNAse and DNAse digestion according to the experimental outline of FIG. 2.

FIG. 4 demonstrates RNA tagging during cDNA synthesis and pickup in reads from next generation sequencing.

FIG. 5A-5B demonstrates that the RNA and DNA libraries have different insert size distributions, typical for their nucleic acid content.

FIG. 6 demonstrates that separated tagged and non-tagged reads show expected mapping to genomic features.

FIG. 7 also demonstrates that separated tagged and non-tagged reads show expected mapping to genomic features.

FIG. 8A-8B provide an example of separated tagged reads mapping to the expected strand of the annotated mRNA genes.

### DETAILED DESCRIPTION OF THE INVENTION

The present invention provides molecular methods to distinguish DNA and RNA in an isolate or a mixture comprising both, by specifically modifying and detecting either the RNA component or the DNA component. In some embodiments, the RNA component is modified and subjected to further analyses, while the DNA component remains unmodified. In alternative embodiments, the DNA component is modified and subjected to further analyses, while the RNA component remains unmodified.

In a particular embodiment, the methods are directed to distinguishing RNA from DNA in a combined isolation by using primers during reverse transcription. Applicants have surprisingly found that known sequence motifs via primers in reverse transcription can be used to distinguish RNA and DNA in a mixture of nucleic acids.

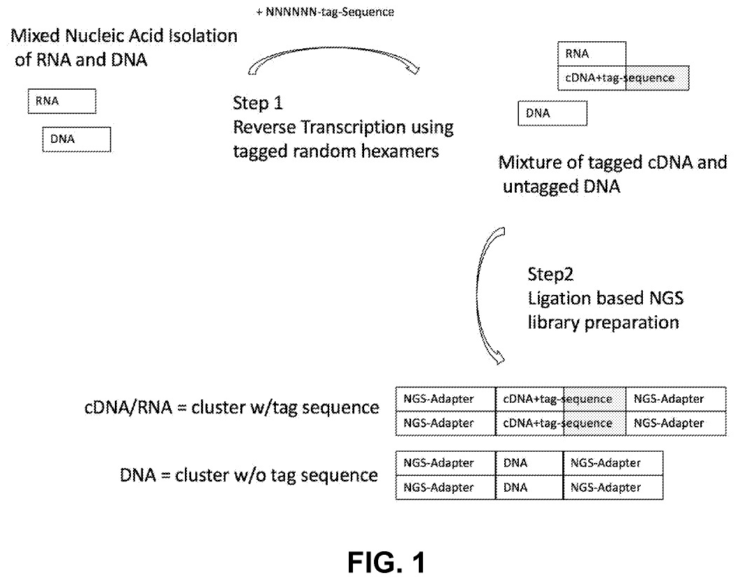

In an embodiment, the methods herein take advantage of the properties of the reverse transcription reaction to distinguish RNA and DNA molecules in NGS datasets by using primers during first strand synthesis, which are modified with a known and unique nucleic acid sequence (“tag”). This is exemplified with reference to FIG. 1, which depicts RNA tagging during reverse transcription. In Step 1, reverse transcription is initiated using, e.g., tagged random hexamers or a mixture of non-random and random sequence, in order to generate tagged cDNAs of the complete transcriptome during reverse transcription, as well as untagged DNA. In Step 2, a ligation based NGS library is prepared to provide cDNA/RNA clusters having tagged sequences, and DNA clusters without tagged sequences.

According to an embodiment, the selective modification of a nucleic acid component includes the use of modified nucleotides during reverse transcription, chemical modification of the RNA component (or the DNA component, depending on the desirability of outcome), to modify nucleotides before transcription.

According to an embodiment, the selective modification of a nucleic acid component includes the use of chemical modification of the RNA component (or the DNA component as relevant) to modify nucleotides before transcription.

According to an embodiment, the selective modification of a nucleic acid component includes the use of one or more natural properties of the reverse transcription enzyme like the incorporation of non-templated nucleotides or known patterns for misincorporation of natural nucleotides to target either the RNA component or the DNA component, respectively.

In an embodiment, the invention provides an assay to evaluate RNA expression and distinguish mutations between RNA and DNA, for example using the whole nucleic acids from samples without the need to separate RNA and DNA via, e.g., different isolation techniques. Exemplary molecular assays include RT-qPCR, next generation library preparation, and other highly sensitive techniques.

Depending on the assay methodology as described herein, an optimal concentration may be required to achieve efficient tagging, sizing and conversion of the cDNA.

In other embodiments, the invention also includes various uses of methods of isolating extracellular vesicles and sequencing nucleic acids from a biological sample for (i) aiding in the diagnosis of a subject, (ii) monitoring the progress or reoccurrence of a disease or other medical condition in a subject, or (iii) aiding in the evaluation of treatment efficacy for a subject undergoing or contemplating treatment for a disease or other medical condition; wherein the presence or absence of one or more biomarkers in the nucleic acid extraction obtained from the method is determined, and the one or more biomarkers are associated with the diagnosis, progress or reoccurrence, or treatment efficacy, respectively, of a disease or other medical condition.

The present invention can be useful in a variety of settings. The overall capability is to simultaneously perform a transcriptome analysis and genomic or epigenomic analysis of a physical sample, and doing so without losing any potentially precious material. A specific example may be the use of RNA expression in combination with the DNA abundance to strengthen the analysis of copy number variation, such as the measurement of chromosomal rearrangements like copy number variations in a given sample. Another application is the simultaneous analysis or measurement of mutations in the RNA and DNA for the use of identifying expressed, relevant tumor neoantigens. Also, simultaneous detection of mutations and other genomic or transcriptomic variants in the RNA and the DNA fraction can lead to an increased sensitivity of detection and a larger panel of identifiable variants within the same molecular assay. Simultaneous mutation analysis in RNA and DNA can also provide representative information of living versus dying cells.

The methods provided herein are useful for detection of rare mutations in blood, as the method provides a sufficiently sensitive method that can be applied on nucleic acids of sufficient amount. The amount of actual DNA and RNA molecules in biofluids is very limited, and the methods provide an isolation method that extracts all molecules of the blood that are relevant for mutation detection in a volume small enough for effective downstream processing and/or analysis.

Where the sample is isolated from exosomes, the method steps herein prepare nucleic acids (RNA and/or DNA) for sequencing. This enables a wide diversity of RNAs and/or DNAs, to be efficiently detected. These can then be used to identify various attributes such as gene expression, alternative splicing, fusion transcripts, circular RNA and the detection of both somatic and germline mutations including single nucleotide variants (SNV) and structural variations (insertions/deletions, fusions, inversions).

As used herein, the term “nucleic acids” refer to DNA and RNA. The nucleic acids can be single stranded or double stranded. In some instances, the nucleic acid is DNA. In some instances, the nucleic acid is RNA. RNA includes, but is not limited to, total RNA, messenger RNA, transfer RNA, ribosomal RNA, non-coding RNAs, microRNAs, and HERV elements. There is a wide diversity of RNA, including, without limitation, ribosomal RNA, SINE RNA, LINE RNA, Alu RNA, HERVs, globin RNA, as well as other types of long non-coding RNAs, small RNAs and/or repeat sequences as described elsewhere, such as at gencodegenes.org/gencode_biotypes.html.

As used herein, the term “biological sample” refers to a sample that contains biological materials such as DNA, RNA and protein. The sample, e.g., biological sample, subjected to the methods described herein may come from any number of sources. The biological sample can be in a fresh or frozen form, and can include cultured cells.

In some embodiments, the biological sample may suitably comprise a bodily fluid from a subject. The bodily fluids can be fluids isolated from anywhere in the body of the subject, such as, for example, a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, intra-organ system fluid, ascitic fluid, tumor cyst fluid, amniotic fluid and cell culture supernatant, and combinations thereof. Biological samples can also include fecal or cecal samples, or supernatants isolated therefrom.

In some embodiments, the biological sample may suitably comprise cell culture supernatant.

In some embodiments, the biological sample may suitably comprise a tissue sample from a subject. The tissue sample can be isolated from anywhere in the body of the subject, and can include fresh tissue, frozen tissue, or even formalin-fixed paraffin embedded (FFPE) tissue.

A suitable sample volume of a bodily fluid is, for example, in the range of about 0.1 mL to about 30 mL fluid. The volume of fluid may depend on a few factors, e.g., the type of fluid used. For example, the volume of serum samples may be about 0.1 mL to about 4 mL, for example, in some embodiments, about 0.2 mL to 4 mL. The volume of plasma samples may be about 0.1 mL to about 4 mL, for example, in some embodiments, 0.5 mL to 4 mL. The volume of urine samples may be about 10 mL to about 30 mL, for example, in some embodiments, about 20 mL.

While the examples provided herein are provided in the context of plasma samples, the skilled artisan will appreciate that these methods are applicable to a variety of biological samples.

The methods and kits of the disclosure are suitable for use with samples derived from a human subject. In addition, the methods and kits of the disclosure are suitable for use with samples derived from a non-human subject such as, for example, a rodent, a non-human primate, a companion animal (e.g., cat, dog, horse), and/or a farm animal (e.g., chicken).

The term “subject” is intended to include all animals shown to or expected to have nucleic acid-containing particles. In particular embodiments, the subject is a mammal, a human or nonhuman primate, a dog, a cat, a horse, a cow, other farm animals, or a rodent (e.g. mice, rats, guinea pig. Etc.). A human subject may be a normal human being without observable abnormalities, e.g., a disease. A human subject may be a human being with observable abnormalities, e.g., a disease. The observable abnormalities may be observed by the human being himself, or by a medical professional. The term “subject,” “patient,” and “individual” are used interchangeably herein.

Also contemplated are biological samples derived from microvesicles, also known as exosomes. Microvesicles are shed by eukaryotic cells, or budded off of the plasma membrane, to the exterior of the cell. These membrane vesicles are heterogeneous in size with diameters ranging from about 10 nm to about 5000 nm. All membrane vesicles shed by cells <0.8 μm in diameter are referred to herein collectively as “extracellular vesicles” or “microvesicles.” These extracellular vesicles include microvesicles, microvesicle-like particles, prostasomes, dexosomes, texosomes, ectosomes, oncosomes, apoptotic bodies, retrovirus-like particles, and human endogenous retrovirus (HERV) particles. Small microvesicles (approximately 10 to 1000 nm, and more often 30 to 200 nm in diameter) that are released by exocytosis of intracellular multivesicular bodies are referred to in the art as “microvesicles.” Exosomes are known to contain RNA types including mRNA (messenger RNA) and miRNA (micro RNA).

Recent studies reveal that nucleic acids within microvesicles have a role as biomarkers. For example, WO 2009/100029 describes, among other things, the use of nucleic acids extracted from microvesicles in GBM patient serum for medical diagnosis, prognosis and therapy evaluation. WO 2009/100029 also describes the use of nucleic acids extracted from microvesicles in human urine for the same purposes. The use of nucleic acids extracted from microvesicles is considered to potentially circumvent the need for biopsies, highlighting the enormous diagnostic potential of microvesicle biology (Skog et al., 2008).

The quality or purity of the isolated extracellular vesicles can directly affect the quality of the extracted extracellular vesicle nucleic acids, which then directly affects the efficiency and sensitivity of biomarker assays for disease diagnosis, prognosis, and/or monitoring. Given the importance of accurate and sensitive diagnostic tests in the clinical field, methods for isolating highly enriched extracellular vesicle fractions from biological samples are needed. Highly enriched extracellular vesicle fractions isolated from biological samples by are obtainable, and high quality nucleic acids can subsequently be extracted from the highly enriched extracellular vesicle fractions. These extracted high quality nucleic acids are useful for measuring or assessing the presence or absence of biomarkers for aiding in the diagnosis, prognosis, and/or monitoring of diseases or other medical conditions.

High quality RNA extractions are desirable because RNA degradation can adversely affect downstream assessment of the extracted RNA, such as in gene expression and mRNA analysis, as well as in analysis of non-coding RNA such as small RNA and microRNA. The methods described herein enable one to extract high quality nucleic acids from extracellular vesicles isolated from a biological sample so that an accurate analysis of nucleic acids within the extracellular vesicles can be performed.

Following the isolation of extracellular vesicles from a biological sample, nucleic acid may be extracted from the isolated or enriched extracellular vesicle fraction. To achieve this, the extracellular vesicles may first be lysed. The lysis of extracellular vesicles and extraction of nucleic acids may be achieved with various methods known in the art, including those described in PCT Publication Nos. WO 2016/007755 and WO 2014/107571, the contents of each of which are hereby incorporated by reference in their entirety. Nucleic acid extraction may be achieved using protein precipitation according to standard procedures and techniques known in the art. Such methods may also utilize a nucleic acid-binding column to capture the nucleic acids contained within the extracellular vesicles. Once bound, the nucleic acids can then be eluted using a buffer or solution suitable to disrupt the interaction between the nucleic acids and the binding column, thereby eluting the nucleic acids.

Starting with a biological sample as described herein, e.g., human plasma, serum, blood, urine, cerebrospinal fluid, and the like, nucleic acids are isolated from exosomes and other cell-free sources. Alternatively, the nucleic acids can originate from tissue sources such as reference standards and formalin-fixed paraffin embedded (FFPE) materials.

Exosomal derived nucleic acids can include RNA or DNA, either individually or as a mixture of RNA and DNA. Exosomal derived nucleic acids can include material either contained within or bound to the outer surface of exosomes. The DNA component can be exosomal or other cell-free sources (cfDNA).

Several methods of isolating microvesicles from a biological sample have been described in the art. For example, a method of differential centrifugation is described in a paper by Raposo et al. (Raposo et al., 1996), a paper by Skog et. al. (Skog et al., 2008) and a paper by Nilsson et. al. (Nilsson et al., 2009). Methods of ion exchange and/or gel permeation chromatography are described in U.S. Pat. Nos. 6,899,863 and 6,812,023. Methods of sucrose density gradients or organelle electrophoresis are described in U.S. Pat. No. 7,198,923. A method of magnetic activated cell sorting (MACS) is described in a paper by Taylor and Gercel Taylor (Taylor and Gercel-Taylor, 2008). A method of nanomembrane ultrafiltration concentration is described in a paper by Cheruvanky et al. (Cheruvanky et al., 2007). A method of Percoll gradient isolation is described in a publication by Miranda et al. (Miranda et al., 2010). Further, microvesicles may be identified and isolated from bodily fluid of a subject by a microfluidic device (Chen et al., 2010). In research and development, as well as commercial applications of nucleic acid biomarkers, it is desirable to extract high quality nucleic acids from biological samples in a consistent, reliable, and practical manner.

In some embodiments, the sample isolation and analysis techniques encompass the methods referred to as EXO50 and/or EXO52 as described in, e.g., WO 2014/107571 and WO 2016/007755, each incorporated by reference herein in the entirety. Also contemplated are the commercially available liquid biopsy platforms sold under the trademarks EXOLUTION™, EXOLUTION PLUS™, EXOLUTION™ UPREP, EXOLUTION HT™, UPREP™, EXOEASY™, EXORNEASY™, each available from Exosome Diagnostics, Inc., as well as the QIAamp Circulating Nucleic Acids Kit, DNeasy Blood & Tissue Kits, AllPrep DNA/RNA Mini Kit, and the AllPrep DNA/RNA/Protein Mini Kit, each available from Qiagen.

Where an extracellular vesicle fraction is utilized, isolation and extraction of nucleic acids, e.g., DNA and/or DNA and nucleic acids including at least RNA from a sample using the following general procedure. First, the nucleic acids in the sample, e.g., the DNA and/or the DNA and the extracellular vesicle fraction, are bound to a capture surface such as a membrane filter, and the capture surface is washed. Then, an elution reagent is used to perform on-membrane lysis and release of the nucleic acids, e.g., DNA and/or DNA and RNA, thereby forming an eluate. The eluate is then contacted with a protein precipitation buffer that includes a transition metal and a buffering agent. The cfDNA and/or DNA and nucleic acids include at least the RNA from the extracellular vesicles is then isolated from the protein-precipitated eluate using any of a variety of art-recognized techniques, such as, for example, binding to a silica column followed by washing and elution.

The elution buffer may comprise a denaturing agent, a detergent, a buffer substance, and/or combinations thereof to maintain a defined solution pH. The elution buffer may include a strong denaturing agent, or even a strong denaturing agent and a reduction agent.

The isolation methods for exosomes for the further purification of extracellular vesicles having associated nucleic acids described herein also include: 1) Ultracentrifugation, often in combination with sucrose density gradients or sucrose cushions to float the relatively low-density exosomes. Isolation of exosomes by sequential differential centrifugations, combined with sucrose gradient ultracentrifugation, can provide high enrichment of exosomes. 2) The use of volume-excluding polymer selected from the group consisting of polyethylene glycol, dextran, dextran sulfate, dextran acetate, polyvinyl alcohol, polyvinyl acetate, or polyvinyl sulfate; and wherein the molecular weight of the volume-excluding polymer is from 1000 to 35000 daltons performed in conjunction with the additive sodium chloride from 0-1M. 3) Size exclusion chromatography, for example, Sephadex™ G200 column matrix. 4) Selective immunoaffinity or charge-based capture using paramagnetic beads (including immuno-precipitation), for example, by using antibodies directed against the surface antigens including but not limited to EpCAM, CD326, KSA, TROP1. The selection antibodies can be conjugated to paramagnetic microbeads. 5) Direct precipitation with chaotropic agents such as guanidinium thiocyanate.

Isolation of microvesicles is contemplated via a membrane as the capture surface, although it should be understood that the format of the capturing surface, e.g., beads or a filter (also referred to herein as a membrane), does not affect the ability of the methods provided herein to efficiently capture extracellular vesicles from a biological sample. A wide range of surfaces are capable of capturing extracellular vesicles as has been reported, but not all surfaces will capture extracellular vesicles (some surfaces do not capture anything).

In embodiments where the capture surface is a membrane, the device for isolating the extracellular vesicle fraction from a biological sample contains at least one membrane. In some embodiments, the device comprises one, two, three, four, five or six membranes. In some embodiments, the device comprises three membranes. In embodiments where the device comprises more than one membrane, the membranes are all directly adjacent to one another at one end of the column. In embodiments where the device comprises more than one membrane, the membranes are all identical to each other, i.e., are of the same charge and/or have the same functional group.

It should be noted that capture by filtering through a pore size smaller than the extracellular vesicles is not the primary mechanism of capture by the methods provided herein. However, filter pore size is nevertheless very important, e.g. because mRNA gets stuck on a 20 nm filter and cannot be recovered, whereas microRNAs can easily be eluted off, and e.g. because the filter pore size is an important parameter in available surface capture area.

The methods provided herein use samples isolated by any of a variety of capture surfaces. In some embodiments, the capture surface is a membrane, also referred to herein as a filter or a membrane filter. In some embodiments, the capture surface is a commercially available membrane. In some embodiments, the capture surface is a charged commercially available membrane. In some embodiments, the capture surface is neutral. In some embodiments, the capture surface is selected from Mustang® Ion Exchange Membrane from PALL Corporation; Vivapure® Q membrane from Sartorius AG; Sartobind Q, or Vivapure® Q Maxi H; Sartobind® D from Sartorius AG, Sartobind (S) from Sartorius AG, Sartobind® Q from Sartorius AG, Sartobind® IDA from Sartorius AG, Sartobind® Aldehyde from Sartorius AG, Whatman® DE81 from Sigma, Fast Trap Virus Purification column from EMD Millipore; Thermo Scientific* Pierce Strong Cation and Anion Exchange Spin Columns.

In embodiments where the capture surface is charged, the capture surface can be a charged filter selected from the group consisting of 0.65 um positively charged Q PES vacuum filtration (Millipore), 3-5 um positively charged Q RC spin column filtration (Sartorius), 0.8 um positively charged Q PES homemade spin column filtration (Pall), 0.8 um positively charged Q PES syringe filtration (Pall), 0.8 um negatively charged S PES homemade spin column filtration (Pall), 0.8 um negatively charged S PES syringe filtration (Pall), and 50 nm negatively charged nylon syringe filtration (Sterlitech). In some embodiments, the charged filter is not housed in a syringe filtration apparatus, as nucleic acid can be harder to get out of the filter in these embodiments. In some embodiments, the charged filter is housed at one end of a column.

In embodiments where the capture surface is a membrane, the membrane can be made from a variety of suitable materials. In some embodiments, the membrane is polyethersulfone (PES) (e.g., from Millipore or PALL Corp.). In some embodiments, the membrane is regenerated cellulose (RC) (e.g., from Sartorius or Pierce).

In some embodiments, the capture surface is a positively charged membrane. In some embodiments, the capture surface is a Q membrane, which is a positively charged membrane and is an anion exchanger with quaternary amines. For example, the Q membrane is functionalized with quaternary ammonium, R—CH₂—N⁺(CH₃)₃. In some embodiments, the capture surface is a negatively charged membrane. In some embodiments, the capture surface is an S membrane, which is a negatively charged membrane and is a cation exchanger with sulfonic acid groups. For example, the S membrane is functionalized with sulfonic acid, R—CH₂—SO₃⁻. In some embodiments, the capture surface is a D membrane, which is a weak basic anion exchanger with diethylamine groups, R—CH₂—NH⁺(C₂H₅)₂. In some embodiments, the capture surface is a metal chelate membrane. For example, the membrane is an IDA membrane, functionalized with minodiacetic acid—N(CH₂COOH⁻)₂. In some embodiments, the capture surface is a microporous membrane, functionalized with aldehyde groups, —CHO. In other embodiments, the membrane is a weak basic anion exchanger, with diethylaminoethyl (DEAE) cellulose. Not all charged membranes are suitable for use in the methods provided herein, e.g., RNA isolated using Sartorius Vivapure S membrane spin column showed RT-qPCR inhibition and, thus, unsuitable for PCR related downstream assay.

In embodiments where the capture surface is charged, extracellular vesicles can be isolated with a positively charged filter.

In embodiments where the capture surface is charged, the pH during extracellular vesicle capture is a pH≤7. In some embodiments, the pH is greater than 4 and less than or equal to 8.

In embodiments where the capture surface is a positively charged Q filter, the buffer system includes a wash buffer comprising 250 mM Bis Tris Propane, pH6.5-7.0. In embodiments where the capture surface is a positively charged Q filter, the lysis buffer is a GTC-based reagent. In embodiments where the capture surface is a positively charged Q filter, the lysis buffer is present at one volume. In embodiments where the capture surface is a positively charged Q filter, the lysis buffer is present at more than one volume.

Depending on the membrane material, the pore sizes of the membrane range from 3 μm to 20 nm. For example, in embodiments where the capture surface is a commercially available PES membrane, the membrane has a pore size of 20 nm (Exomir), 0.65 μm (Millipore) or 0.8 μm (Pall). In embodiments where the capture surface is a commercially available RC membrane, the membrane has a pore size in the range of 3-5 μm (Sartorius, Pierce).

The surface charge of the capture surface can be positive, negative or neutral. In some embodiments, the capture surface is a positively charged bead or beads.

The methods herein are also useful when combined with quick and easy isolation of nucleic acid-containing particles from biological samples such as body fluids and extraction of high quality nucleic acids from the isolated particles. The methods may be suitable for adaptation and incorporation into a compact device or an semi- or fully-automated instrument for use in a laboratory or clinical setting, or in the field.

In some embodiments, the sample is not pre-processed prior to isolation and extraction of nucleic acids, e.g., DNA and/or DNA and RNA, from the biological sample.

In some embodiments, the sample is subjected to a pre-processing step prior to isolation, purification or enrichment of the extracellular vesicles is performed to remove large unwanted particles, cells and/or cell debris and other contaminants present in the biological sample. The pre-processing steps may be achieved through one or more centrifugation steps (e.g., differential centrifugation) or one or more filtration steps (e.g., ultrafiltration), or a combination thereof. Where more than one centrifugation pre-processing steps are performed, the biological sample may be centrifuged first at the lower speed and then at the higher speed. If desired, further suitable centrifugation pre-processing steps may be carried out. Alternatively, or in addition to the one or more centrifugation pre-processing steps, the biological sample may be filtered. For example, a biological sample may be first centrifuged at 20,000 g for 1 hour to remove large unwanted particles; the sample can then be filtered, for example, through a 0.8 μm filter.

In some embodiments, the sample is pre-filtered to exclude particles larger than 0.8 μm. In some embodiments, the sample includes an additive such as EDTA, sodium citrate, and/or citrate-phosphate-dextrose. In some embodiments, the sample does not contain heparin, as heparin can negatively impact RT-qPCR and other nucleic acid analysis. In some embodiments, the sample is mixed with a buffer prior to purification and/or nucleic acid isolation and/or extraction. In some embodiments, the buffer is a binding buffer.

In some embodiments, one or more centrifugation steps are performed before or after contacting the biological sample with the capture surface to separate extracellular vesicles and concentrate the extracellular vesicles isolated from the biological fraction. To remove large unwanted particles, cells, and/or cell debris, the samples may be centrifuged at a low speed of about 100-500 g, for example, in some embodiments, about 250-300 g. Alternatively or in addition, the samples may be centrifuged at a higher speed. Suitable centrifugation speeds are up to about 200,000 g; for example, from about 2,000 g to less than about 200,000 g. Speeds of above about 15,000 g and less than about 200,000 g or above about 15,000 g and less than about 100,000 g or above about 15,000 g and less than about 50,000 g are used in some embodiments. Speeds of from about 18,000 g to about 40,000 g or about 30,000 g; and from about 18,000 g to about 25,000 g are more preferred. In some embodiments, a centrifugation speed of about 20,000 g. Generally, suitable times for centrifugation are from about 5 minutes to about 2 hours, for example, from about 10 minutes to about 1.5 hours, or from about 15 minutes to about 1 hour. A time of about 0.5 hours may be used. It is sometimes useful, in some embodiments, to subject the biological sample to centrifugation at about 20,000 g for about 0.5 hours. However, the above speeds and times can suitably be used in any combination (e.g., from about 18,000 g to about 25,000 g, or from about 30,000 g to about 40,000 g for about 10 minutes to about 1.5 hours, or for about 15 minutes to about 1 hour, or for about 0.5 hours, and so on). The centrifugation step or steps may be carried out at below-ambient temperatures, for example at about 0-10° C., for example, about 1-5° C., e.g., about 3° C. or about 4° C.

In some embodiments, one or more filtration steps are performed before or after contacting the biological sample with the capture surface. A filter having a size in the range about 0.1 to about 1.0 μm may be employed, for example, about 0.8 μm or 0.22 μm. The filtration may also be performed with successive filtrations using filters with decreasing porosity.

In some embodiments, one or more concentration steps are performed, in order to reduce the volumes of sample to be treated during the chromatography stages, before or after contacting the biological sample with the capture surface. Concentration may be through centrifugation of the sample at high speeds, e.g. between 10,000 and 100,000 g, to cause the sedimentation of the extracellular vesicles. This may consist of a series of differential centrifugations. The extracellular vesicles in the pellet obtained may be reconstituted with a smaller volume and in a suitable buffer for the subsequent steps of the process. The concentration step may also be performed by ultrafiltration. In fact, this ultrafiltration both concentrates the biological sample and performs an additional purification of the extracellular vesicle fraction. In another embodiment, the filtration is an ultrafiltration, for example, a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibers (Amicon, Millipore, Sartorius, Pall, GF, Sepracor). Within the scope of the invention, the use of membranes with a cut-off threshold below 1000 kDa, for example, in some embodiments, between 100 kDa and 1000 kDa, or for example, in some embodiments, between 100 kDa and 600 kDa, is advantageous.

In some embodiments, one or more size-exclusion chromatography step or gel permeation chromatography steps are performed before or after contacting the biological sample with the capture surface. To perform the gel permeation chromatography step, a support selected from silica, acrylamide, agarose, dextran, ethylene glycol-methacrylate co-polymer or mixtures thereof, e.g., agarose-dextran mixtures, are used in some embodiments. For example, such supports include, but are not limited to: SUPERDEX® 200HR (Pharmacia), TSK G6000 (TosoHaas) or SEPHACRYL® S (Pharmacia).

In some embodiments, one or more affinity chromatography steps are performed before or after contacting the biological sample with the capture surface. Some extracellular vesicles can also be characterized by certain surface molecules. Because microvesicles form from budding of the cell plasma membrane, these microvesicles often share many of the same surface molecules found on the cells they originated from. As used herein, “surface molecules” refers collectively to antigens, proteins, lipids, carbohydrates, and markers found on the surface or in or on the membrane of the microvesicle. These surface molecules can include, for example, receptors, tumor-associated antigens, membrane protein modifications (e.g., glycosylated structures). For example, microvesicles that bud from tumor cells often display tumor-associated antigens on their cell surface. As such, affinity chromatography or affinity exclusion chromatography can also be utilized in combination with the methods provided herein to isolate, identify, and or enrich for specific populations of microvesicles from a specific donor cell type (Al-Nedawi et al., 2008; Taylor and Gercel-Taylor, 2008). For example, tumor (malignant or non-malignant) microvesicles carry tumor-associated surface antigens and may be detected, isolated and/or enriched via these specific tumor-associated surface antigens. In one example, the surface antigen is epithelial cell adhesion molecule (EpCAM), which is specific to microvesicles from carcinomas of lung, colorectal, breast, prostate, head and neck, and hepatic origin, but not of hematological cell origin (Balzar et al., 1999; Went et al., 2004). Additionally, tumor-specific microvesicles can also be characterized by the lack of certain surface markers, such as CD80 and CD86. In these cases, microvesicles with these markers may be excluded for further analysis of tumor specific markers, e.g., by affinity exclusion chromatography. Affinity chromatography can be accomplished, for example, by using different supports, resins, beads, antibodies, aptamers, aptamer analogs, molecularly imprinted polymers, or other molecules known in the art that specifically target desired surface molecules on microvesicles.

In some embodiments, one or more control particles or one or more nucleic acid(s) may be added to the sample prior to extracellular vesicle isolation and/or nucleic acid extraction to serve as an internal control to evaluate the efficiency or quality of extracellular vesicle purification and/or nucleic acid extraction. The methods described herein provide for the efficient isolation and the control nucleic acid(s) along with the extracellular vesicle fraction. These control nucleic acid(s) include one or more nucleic acids from Q-beta bacteriophage, one or more nucleic acids from virus particles, or any other control nucleic acids (e.g., at least one control target gene) that may be naturally occurring or engineered by recombinant DNA techniques. In some embodiments, the quantity of control nucleic acid(s) is known before the addition to the sample. The control target gene can be quantified using real-time PCR analysis. Quantification of a control target gene can be used to determine the efficiency or quality of the extracellular vesicle purification or nucleic acid extraction processes.

In some embodiments, the control nucleic acid is a nucleic acid from a Q-beta bacteriophage, referred to herein as “Q-beta control nucleic acid.” The Q-beta control nucleic acid used in the methods described herein may be a naturally-occurring virus control nucleic acid or may be a recombinant or engineered control nucleic acid. Q-beta is a member of the leviviridae family, characterized by a linear, single-stranded RNA genome that consists of 3 genes encoding four viral proteins: a coat protein, a maturation protein, a lysis protein, and RNA replicase. When the Q-beta particle itself is used as a control, due to its similar size to average microvesicles, Q-beta can be easily purified from a biological sample using the same purification methods used to isolate microvesicles, as described herein. In addition, the low complexity of the Q-beta viral single-stranded gene structure is advantageous for its use as a control in amplification-based nucleic acid assays. The Q-beta particle contains a control target gene or control target sequence to be detected or measured for the quantification of the amount of Q-beta particle in a sample. For example, the control target gene is the Q-beta coat protein gene. When the Q-beta particle itself is used as a control, after addition of the Q-beta particles to the biological sample, the nucleic acids from the Q-beta particle are extracted along with the nucleic acids from the biological sample using the extraction methods described herein. When a nucleic acid from Q-beta, for example, RNA from Q-beta, is used as a control, the Q-beta nucleic acid is extracted along with the nucleic acids from the biological sample using the extraction methods described herein. Detection of the Q-beta control target gene can be determined by RT-PCR analysis, for example, simultaneously with the biomarker(s) of interest. A standard curve of at least 2, 3, or 4 known concentrations in 10-fold dilution of a control target gene can be used to determine copy number. The copy number detected and the quantity of Q-beta particle added or the copy number detected and the quantity of Q-beta nucleic acid, for example, Q-beta RNA, added can be compared to determine the quality of the isolation and/or extraction process.

In some embodiments, 50, 100, 150, 200, 250, 300, 350, 400, 450, 500, 1,000 or 5,000 copies of Q-beta particles or Q-beta nucleic acid, for example, Q-beta RNA, added to a bodily fluid sample. In some embodiments, 100 copies of Q-beta particles or Q-beta nucleic acid, for example, Q-beta RNA, are added to a bodily fluid sample. When the Q-beta particle itself is used as control, the copy number of Q-beta particles can be calculated based on the ability of the Q-beta bacteriophage to infect target cells. Thus, the copy number of Q-beta particles is correlated to the colony forming units of the Q-beta bacteriophage.

Optionally, control particles may be added to the sample prior to extracellular vesicle isolation or nucleic acid extraction to serve as an internal control to evaluate the efficiency or quality of extracellular vesicle purification and/or nucleic acid extraction. The methods described herein provide for the efficient isolation and the control particles along with the extracellular vesicle fraction. These control particles include Q-beta bacteriophage, virus particles, or any other particle that contains control nucleic acids (e.g., at least one control target gene) that may be naturally occurring or engineered by recombinant DNA techniques. In some embodiments, the quantity of control particles is known before the addition to the sample. The control target gene can be quantified using real-time PCR analysis. Quantification of a control target gene can be used to determine the efficiency or quality of the extracellular vesicle purification or nucleic acid extraction processes.

In some embodiments, the Q-beta particles are added to the urine sample prior to nucleic extraction. For example, the Q-beta particles are added to the urine sample prior to ultrafiltration and/or after the pre-filtration step.

In some embodiments, the methods and kits described herein include one or more in-process controls. In some embodiments, the in-process control is detection and analysis of a reference gene that indicates sample quality (i.e., an indicator of the quality of the biological sample, e.g., biofluid sample). In some embodiments, the in-process control is detection and analysis of a reference gene that indicates plasma quality (i.e., an indicator of the quality of the plasma sample). In some embodiments, the reference gene(s) is/are analyzed by additional qPCR.

In some embodiments, the in-process control is an in-process control for reverse transcriptase and/or PCR performance. These in-process controls include, by way of non-limiting examples, a reference RNA (also referred to herein as ref RNA), that is spiked in after RNA isolation and prior to reverse transcription. In some embodiments, the ref RNA is a control such as Qbeta. In some embodiments, the ref RNA is analyzed by additional PCR.

In some embodiments, a spike-in of synthetic RNA or DNA standard, also referred to herein as a “synthetic spike-in” is included as a quality control metric, or at any step prior to sequencing library preparation. Exogenous materials such as synthetic nucleic acids, can serve as sample quality control reagents, quantification reagents, can enable limit of detection, dynamic range and technical reproducibility studies and/or can enable studies detecting particular sequences.

Commercially available synthetic spike-ins include, without limitation, Dharmacon: Solaris RNA spike-in control kit; Exiqon: RNA spike-in kit; Horizon Diagnostics: Reference standards, Lexogen: spike-in RNA variant control mixes; Thermo Fisher Scientific: ERCC RNA spike-in control mixes; and Qbeta RNA spike-in, yeast or Arabidopsis RNA.

In some embodiments, the synthetic spike-ins is added to the sample at different dilutions. In some embodiments, the dilution of the spike-ins to be added to the sample can be in the range of 1:1000 to 1:10,000,000, including, without limitation, dilutions of 1:1000, 1:10,000, 1:100,000, 1:1,000,000 and even 1:10,000,000. The specific dilution of spike-ins to be added to the sample is determined based on the quantity and/or the quality and/or source of the nucleic acids present in the sample.

The invention provides methods utilizing a selective modification of nucleic acid components in a given mixture.

As noted, a variety of techniques are contemplated to accomplish this modification, including, the use of modified nucleotides during reverse transcription, chemical modification of the RNA to modify nucleotides, use of natural properties of the reverse transcription enzyme, such as the incorporation of non-templated nucleotides, or known patterns of mis-incorporation of natural nucleotides.

An exemplary modification technique uses properties of the reverse transcription reaction to distinguish RNA and DNA molecules present in a combined preparation of total nucleic acids. In a particular embodiment, the invention provides a method for distinguishing RNA from DNA in a combined preparation of total nucleic acids by using primers tagged with a known nucleic acid sequence during reverse transcription.

The inventive methods are useful in an assay to evaluate RNA expression and distinguish mutations between RNA and DNA, by utilizing the whole nucleic acids from samples without a need to separate RNA and DNA, e.g., via different isolation techniques.

In an embodiment, the invention provides methods to distinguish RNA from DNA in assays downstream of isolation using modification of the reverse transcription reaction, a technique which converts RNA molecules into copies of complementary DNA molecules (cDNA).

In an embodiment, the modification uses known DNA sequences (sequence tags) to simultaneously start (“prime”) the reverse transcription reaction and mark the cDNA as derived from an RNA precursor (tagged cDNA). This allows various types of assay methodology and algorithms to distinguish sequences originating from RNA, as compared to sequences originating from DNA.

According to a particular embodiment, the high specificity of the reverse transcriptase enzyme for RNA as a template is used to tag the resulting cDNA in order to distinguish it from DNA.

For tagging of cDNA and to prime the reverse transcriptase reaction, a variety of sequences can be used. Exemplary are specific sequences, random sequences, and mixtures thereof, xenobiotic sequences, molecular identifiers, human sequences, random hexamers, or tagged random nucleotides of other length.

Exemplary tagged primers include, e.g., oligonucleotide that comprises random nucleotides, such as tagged random hexamers, alone or in combination with nonrandom nucleotides.

The nonrandom nucleotide portion of an oligonucleotide can be designed to provide an efficient substrate for reverse transcription and provide means to be identified in a downstream procedure, including features such as (1) a balanced G-C content, such as a G-C content in the range of about 40-60%, such as about 40-50%, or even about 40-45%, (2), a nucleic acid sequence that is of size the range of about 1-40, 1-20, or even 1-5 nucleotides, (3) a nucleic acid sequence that is not present in the genome of interest, such as the human genome, and/or (4) a nucleic acid sequence that is unique to the genome of interest.

The random nucleotide portion of the oligonucleotide can also be selectively randomized, e.g., by depletion of nucleotide combinations known to prime at unwanted locations, e.g. at highly abundant RNAs (e.g. rRNA or repeat elements), highly similar sequences (e.g. pseudogenes) or promiscuous priming sites (e.g. with high G-C content).

In some embodiments, the sample can either be subjected to a reverse transcription reaction or untreated. The RNA within a sample is reverse transcribed when it is of interest to convert the RNA to cDNA. In some embodiments, only first stand synthesis is conducted when only single stranded cDNA is desired. In some embodiments, both first strand and second strand synthesis is conducted when double stranded DNA is desired. In some embodiments, the sample is untreated when it is of interest to only investigate DNA fractions within the sample. In some embodiments, the cDNA processing steps include, for example but not limited to retaining strand information by treating with uracil-N-glycosylase and/or by orientation of NGS adapter sequences, cleavage of RNA, fragmentation of RNA, incorporation of non-canonical nucleotides, annealing or ligation of adapter sequences (adaptor ligation), second strand synthesis, etc.

In some embodiments, the sample is subjected to fragmentation or untreated. Fragmentation can be achieved using enzymatic or non-enzymatic processes or by physical shearing of the material with RNA or dsDNA. In some embodiments, fragmentation of the RNA and/or dsDNA is conducted by heat denaturation in the presence of divalent cations. The specific duration of fragmentation time of the sample is determined based on the quantity and/or the quality and/or source of the nucleic acids present in the sample. In some embodiments, the duration of fragmentation time ranges from 0 minute to 30 minutes.

In some embodiments, sequencing adaptors are added to the material using ligation based approaches following end-repair and adenylation, such as polyadenylation. In some embodiments, sequencing adaptors are added to the material using PCR-based approaches. Nucleic acids within the sample, which have gone through any of the embodiments described above and now have sequence adaptors will hereto be described as ‘library’ when referring to the entire collection of nucleic acid fragments within the sample or ‘library fragment’ when referring to the fragment of nucleic acid that has been incorporated within the context of the sequence adaptors. Inclusion of unique molecular index (UMI), unique identifier, or molecular tag in the adapter sequence provides an added benefit for read de-duplication and enhanced estimation of the input number of nucleic acid molecules in the sample.

In some embodiments, using bead-based separation techniques, the library can be subjected to a process whereby composition of the library can be further modified to: 1) remove unwanted products (including but not restricted to; residual adaptors, primers, buffers, enzymes, adaptor dimers); 2) be of a certain size range (by altering the bead or bead buffer reagent to sample ratio, low and/or high molecular weight products can be either included or excluded in the sample); 3) concentrate the sample by elution in minimal volume. This process is commonly referred to as a ‘clean up’ step or the sample is ‘cleaned up’ and will hereto be referred to as such. Bead-based separation techniques can include but are not limited to paramagnetic beads. Bead-based clean up can be conducted once or multiple times if required or desired.

Commercially available paramagnetic beads useful according to the methods herein include, without limitation, Beckman Coulter: Agencourt AMPure XP; Beckman Coulter: Agencourt RNAclean XP; Kapa Biosystems: Kapa Pure beads; Omega Biosystems: MagBind TotalPure NGS beads; and ThermoFisher Scientific: Dynabeads.

Following bead-based clean up, the library can be amplified en masse using universal primers that target the adaptor sequence. The number of amplification cycles can be modified to produce enough product that is required for downstream processing steps.

Library quantity and quality is quantified using, but not limited to, fluorometric techniques such as Qubit dsDNA HS assay and/or Agilent Bioanalyzer HS DNA assay. The libraries can then be normalized, multiplexed and subjected to sequencing on any next generation sequencing platform.

In some embodiments, the extracted nucleic acid comprises DNA and/or DNA and RNA. In embodiments where the extracted nucleic acid comprises DNA and RNA, the RNA is reverse-transcribed into complementary DNA (cDNA) before further amplification. Such reverse transcription may be performed alone or in combination with an amplification step. One example of a method combining reverse transcription and amplification steps is reverse transcription polymerase chain reaction (RT-PCR), which may be further modified to be quantitative, e.g., quantitative RT-PCR as described in U.S. Pat. No. 5,639,606, which is incorporated herein by reference for this teaching. Another example of the method comprises two separate steps: a first of reverse transcription to convert RNA into cDNA and a second step of quantifying the amount of cDNA using quantitative PCR. As demonstrated in the examples that follow, the RNAs extracted from nucleic acid-containing particles using the methods disclosed herein include many species of transcripts including, but not limited to, ribosomal 18S and 28S rRNA, microRNAs, transfer RNAs, transcripts that are associated with diseases or medical conditions, and biomarkers that are important for diagnosis, prognosis and monitoring of medical conditions.

For example, RT-PCR analysis determines a Ct (cycle threshold) value for each reaction. In RT-PCR, a positive reaction is detected by accumulation of a fluorescence signal. The Ct value is defined as the number of cycles required for the fluorescent signal to cross the threshold (i.e., exceeds background level). Ct values are inversely proportional to the amount of target nucleic acid, or control nucleic acid, in the sample (i.e., the lower the Ct value, the greater the amount of control nucleic acid in the sample).

In another embodiment, the copy number of the control nucleic acid can be measured using any of a variety of art-recognized techniques, including, but not limited to, RT-PCR. Copy number of the control nucleic acid can be determined using methods known in the art, such as by generating and utilizing a calibration, or standard curve.

In some embodiments, one or more biomarkers can be one or a collection of genetic aberrations, which is used herein to refer to the nucleic acid amounts as well as nucleic acid variants within the nucleic acid-containing particles. Specifically, genetic aberrations include, without limitation, over-expression of a gene (e.g., an oncogene) or a panel of genes, under-expression of a gene (e.g., a tumor suppressor gene such as p53 or RB) or a panel of genes, alternative production of splice variants of a gene or a panel of genes, gene copy number variants (CNV) (e.g., DNA double minutes) (Hahn, 1993), nucleic acid modifications (e.g., methylation, acetylation and phosphorylations), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splice variants and/or changes of gene expression level, or combinations of any of the foregoing.

Where reverse transcriptase is performed as described elsewhere herein, the resultant modified components can be integrated with a variety of molecular assay techniques for identification according to the methods herein. Exemplary assays are PCR, qPCR, next generation sequencing, Sanger sequencing, primer extension, cDNA library preparation, adapter ligation, and other molecular methods as described.

The identification and analysis of nucleic acids present is quantitative and/or qualitative. For quantitative analysis, the amounts (expression levels), either relative or absolute, of specific nucleic acids of interest within the isolated particles are measured with methods known in the art (described below). For qualitative analysis, the species of specific nucleic acids of interest within the isolated extracellular vesicles, whether wild type or variants, are identified with methods known in the art.

In some embodiments, it may be beneficial or otherwise desirable to amplify the nucleic acid of the extracellular vesicle prior to analyzing it. Methods of nucleic acid amplification are commonly used and generally known in the art, many examples of which are described herein. If desired, the amplification can be performed such that it is quantitative. Quantitative amplification will allow quantitative determination of relative amounts of the various nucleic acids, to generate a genetic or expression profile.

Nucleic acid amplification methods include, without limitation, polymerase chain reaction (PCR) (U.S. Pat. No. 5,219,727) and its variants such as in situ polymerase chain reaction (U.S. Pat. No. 5,538,871), quantitative polymerase chain reaction (U.S. Pat. No. 5,219,727), nested polymerase chain reaction (U.S. Pat. No. 5,556,773), self-sustained sequence replication and its variants (Guatelli et al., 1990), transcriptional amplification system and its variants (Kwoh et al., 1989), Qb Replicase and its variants (Miele et al., 1983), cold-PCR (Li et al., 2008), BEAMing (Li et al., 2006), or any other nucleic acid amplification methods, followed by the detection of the amplified molecules using techniques well known to those of skill in the art. Especially useful are those detection schemes designed for the detection of nucleic acid molecules if such molecules are present in very low numbers. The foregoing references are incorporated herein for their teachings of these methods. In other embodiment, the step of nucleic acid amplification is not performed. Instead, the extract nucleic acids are analyzed directly (e.g., through next-generation sequencing).

The determination of such genetic aberrations can be performed by a variety of techniques known to the skilled practitioner. For example, expression levels of nucleic acids, alternative splicing variants, chromosome rearrangement and gene copy numbers can be determined by microarray analysis (see, e.g., U.S. Pat. Nos. 6,913,879, 7,364,848, 7,378,245, 6,893,837 and 6,004,755) and quantitative PCR. Particularly, copy number changes may be detected with the Illumina Infinium II whole genome genotyping assay or Agilent Human Genome CGH Microarray (Steemers et al., 2006). Nucleic acid modifications can be assayed by methods described in, e.g., U.S. Pat. No. 7,186,512 and patent publication WO2003/023065. Particularly, methylation profiles may be determined by Illumina DNA Methylation OMA003 Cancer Panel. SNPs and mutations can be detected by hybridization with allele-specific probes, enzymatic mutation detection, chemical cleavage of mismatched heteroduplex (Cotton et al., 1988), ribonuclease cleavage of mismatched bases (Myers et al., 1985), mass spectrometry (U.S. Pat. Nos. 6,994,960, 7,074,563, and 7,198,893), nucleic acid sequencing, single strand conformation polymorphism (SSCP) (Orita et al., 1989), denaturing gradient gel electrophoresis (DGGE) (Fischer and Lerman, 1979a; Fischer and Lerman, 1979b), temperature gradient gel electrophoresis (TGGE) (Fischer and Lerman, 1979a; Fischer and Lerman, 1979b), restriction fragment length polymorphisms (RFLP) (Kan and Dozy, 1978a; Kan and Dozy, 1978b), oligonucleotide ligation assay (OLA), allele-specific PCR (ASPCR) (U.S. Pat. No. 5,639,611), ligation chain reaction (LCR) and its variants (Abravaya et al., 1995; Landegren et al., 1988; Nakazawa et al., 1994), flow-cytometric heteroduplex analysis (WO/2006/113590) and combinations/modifications thereof. Notably, gene expression levels may be determined by the serial analysis of gene expression (SAGE) technique (Velculescu et al., 1995). In general, the methods for analyzing genetic aberrations are reported in numerous publications, not limited to those cited herein, and are available to skilled practitioners. The appropriate method of analysis will depend upon the specific goals of the analysis, the condition/history of the patient, and the specific cancer(s), diseases or other medical conditions to be detected, monitored or treated. The forgoing references are incorporated herein for their teaching of these methods.

Many biomarkers may be associated with the presence or absence of a disease or other medical condition in a subject. Therefore, detection of the presence or absence of a biomarker or combination of biomarkers in a nucleic acid extraction from isolated particles, according to the methods disclosed herein, aid diagnosis of a disease or other medical condition in the subject.

Further, many biomarkers may help disease or medical status monitoring in a subject. Therefore, the detection of the presence or absence of such biomarkers in a nucleic acid extraction from isolated particles, according to the methods disclosed herein, may aid in monitoring the progress or reoccurrence of a disease or other medical condition in a subject.

Many biomarkers have also been found to influence the effectiveness of treatment in a particular patient. Therefore, the detection of the presence or absence of such biomarkers in a nucleic acid extraction from isolated particles, according to the methods disclosed herein, may aid in evaluating the efficacy of a given treatment in a given patient. The identification of these biomarkers in nucleic acids extracted from isolated particles from a biological sample from a patient may guide the selection of treatment for the patient.

In certain embodiments of the foregoing aspects of the invention, the disease or other medical condition is a neoplastic disease or condition (e.g., cancer or cell proliferative disorder).

In some embodiments, the extracted nucleic acids, e.g., exosomal RNA, also referred to herein as “exoRNA,” are further analyzed based on detection of a biomarker or a combination of biomarkers. In some embodiments, the further analysis is performed using machine-learning based modeling, data mining methods, and/or statistical analysis. In some embodiments, the data is analyzed to identify or predict disease outcome of the patient. In some embodiments, the data is analyzed to stratify the patient within a patient population. In some embodiments, the data is analyzed to identify or predict whether the patient is resistant to treatment. In some embodiments, the data is used to measure progression-free survival progress of the subject.

In some embodiments, the data is analyzed to select a treatment option for the subject when a biomarker or combination of biomarkers is detected. In some embodiments, the treatment option is treatment with a combination of therapies.

In some embodiments, “next-generation” sequencing (NGS) or high-throughput sequencing experiments are performed according to the methods of the invention. These sequencing techniques allow for the identification of nucleic acids present in low or high abundance in a sample, or which are otherwise not detected by more conventional hybridization methods. NGS typically incorporate the addition of nucleotides followed by washing steps.

Commercially available kits for total RNA SEQUENCING which preserves the strand information, meant for mammalian RNA and very low input RNA are useful in this regard, and include, without limitation, Clontech: SMARTer stranded total RNASeq kit; Clontech: SMARTSeq v4 ultra low input RNASeq kit; Illumina: Truseq stranded total RNA library prep kit; Kapa Biosystems: Kapa stranded RNASeq library preparation kit; New England Biolabs: NEBNext ultra directional library prep kit; Nugen: Ovation Solo RNASeq kit; and Nugen: Nugen Ovation RNASeq system v2.

### EXAMPLES

While the Examples provided herein use a variety of membranes and devices used for centrifugation and/or filtration purposes, it is to be understood that these methods can be used with any capture surface and/or housing device that allows for the efficient capture of extracellular vesicles and release of the nucleic acids, particularly RNA, contained therein.

Sample Isolation

Samples are generally obtained from commercial sources and isolated by EXOLUTION PLUS™, available from Exosome Diagnostics, Inc., to provide a mixture of exosomal DNA and RNA, along with present cfDNA.

### Example 1

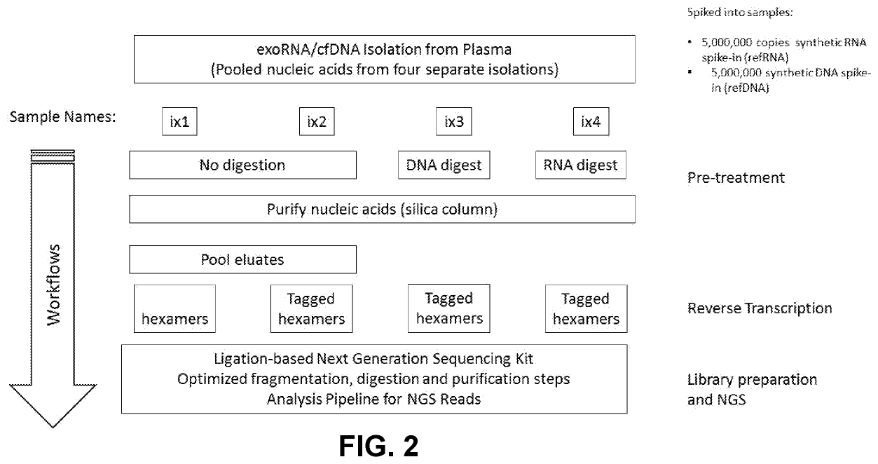

To perform a proof-of-principle experiment demonstrating successful RNA tagging in NGS, combinations of the following steps can be used (1) RNase: RNase A solution 10 mg/ml (# E866-1 ml Amresco) following the manufacturer's instructions (2) DNase: RQ1 RNase-free DNase (# M6101 Promega) following the manufacturer's instructions (3) Cleanup of digestion reactions: using 350 μl RLT buffer (RNeasy All Prep Kit) and 2 volumes of ethanol (760 μL) and following the manufacturer's instructions (4) NGS Library Prep using any combination of First Strand cDNA synthesis, Second Strand Synthesis, End Repair and Adenylation, Adapter Ligation, including Adapters containing a unique molecular barcode used for error reduction and quantification, Library enrichment PCR, Bead-based and Column-based NA Purifications, Cluster Generation and Next Generation Sequencing. This is also illustrated schematically in FIG. 2.

In particular, a suitable experimental setup is outlined that will use a RNAse or DNAse or mock digestion of a mixture of exosomal RNA and cell-free DNA to demonstrate the specificity and efficiency of the RNA tagging process and the detection of the tag in an NGS dataset. Sample ix1 (control) will contain RNA and DNA but use only regular hexamers without a tag in the cDNA synthesis, ix2 (inventive process) will contain RNA and DNA and use tagged hexamers during cDNA synthesis, ix3 (control) will contain only RNA left intact by DNAse digestion and use tagged hexamers during cDNA synthesis, and ix4 (control) will contain DNA left intact by RNase digestion. To demonstrate successful enzymatic digestion in the NGS libraries, 5,000,000 copies of a synthetic RNA and DNA of a specific, unique sequence were spiked into the nucleic acid extraction.

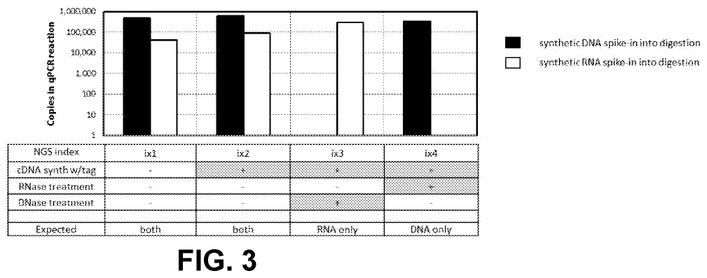

The results are demonstrated in FIG. 3, which shows the successful enzymatic digestion removing either DNA or RNA, the final 100 nM NGS library was diluted 1:20 and 2 μL were subjected to qPCR using a standard curve of reference material to enable absolute quantification. The signals from the synthetic RNA and DNA spike-ins in the four samples demonstrate a successful DNase and RNase digestion, respectively.

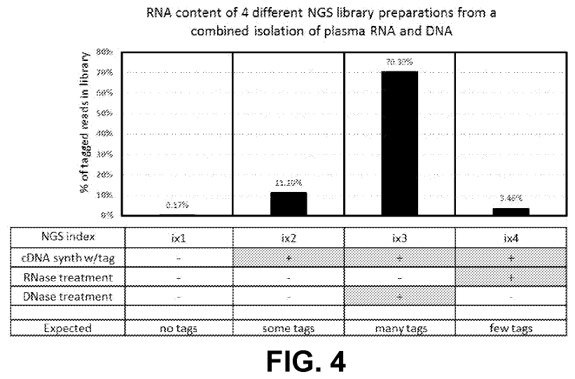

The percent of read-pairs obtained from sequencing that carry a tag sequence using the presented method on a mixture of RNA and DNA (index2 or “ix2”) and three different control samples (index1 or “ix1”, index3 or “ix3”, index4 or “ix4”) are shown in FIG. 4. The sequencing data was mapped to GRCh38 with gencode v25 annotations using STAR. The reads were separated into tagged (RNA) and non-tagged (DNA) based on the presence of the sequence tag with allowing a single mismatch in the 9 bp search sequence found within a 15 bp window from the start of the NGS read. Input to ix3 was DNAse digested and therefore contains many sequence tags. Input to ix4 was RNAse digested and therefore contains little sequence tags. In ix1, where no tags were introduced experimentally, the tags obtained by sequencing match the number that is expected by chance. A more stringent search with 0 MM allowed reduction in the false identification rate from 0.17% to 0.03% without significantly effecting the recovery of tagged RNA reads. This is consistent with the tagged read-pairs originating from RNA and the non-tagged read-pairs originating from DNA.

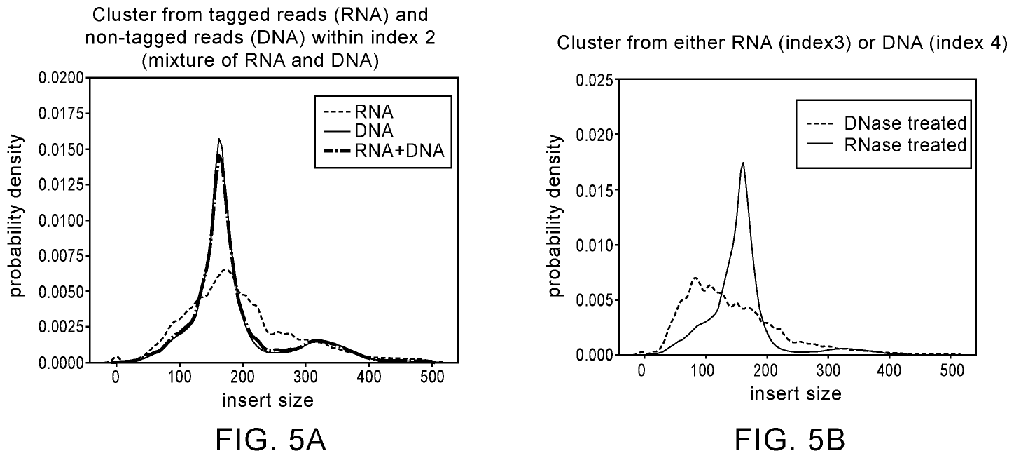

To demonstrate that the tagged reads in the NGS library generated from the mixture of RNA and DNA are indeed RNA, we looked at the typical distribution of insert sizes in the libraries. This is shown in FIG. 5A-5B, which demonstrates that separated tagged and non-tagged reads show expected insert size distribution. In the library from input material consisting of RNA and DNA (index2), RNA reads are separated from DNA reads and aligned to the genome. Insert sizes are plotted. While RNA reads show a typical profile for an RNA library (compare index3, DNAse treated control) the DNA reads show a typical DNA profile (compare index4, RNase treated control). This is consistent with the tagged read-pairs originating from RNA and the non-tagged read-pairs originating from DNA.

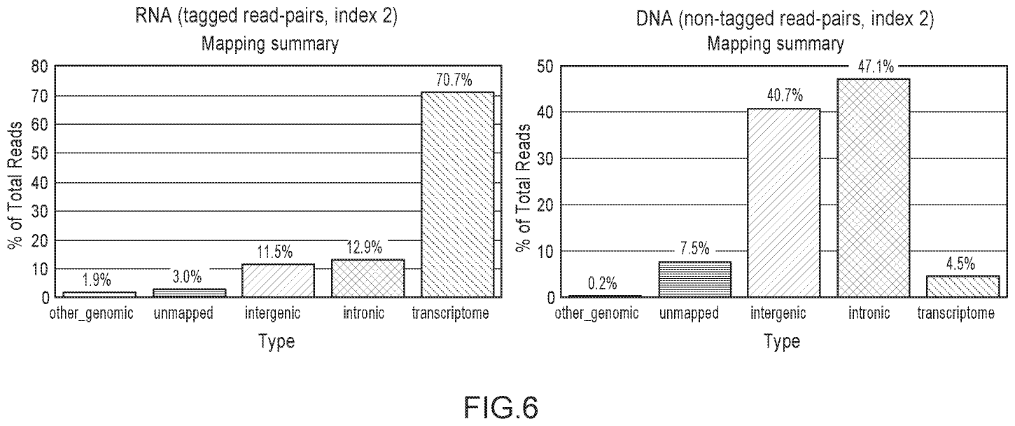

In constructing a library from input material consisting of RNA and DNA, tagged reads are separated from non-tagged reads and aligned to expected mapping of genomic features, as presented in FIG. 6. A read-pair was classified as intergenic or intronic if the position maps more than 1 bp outside of an annotated exon, which can be considered a very strict cut-off to identify only transcriptomic reads. Reads from the tagged library primarily map to transcriptomic features and reads from the non-tagged library primarily map to intergenic and intronic features (e.g., the expected amount of intronic mapping for a DNA library is 46.8%). This is consistent with the tagged read-pairs originating from RNA and the non-tagged read-pairs originating from DNA.

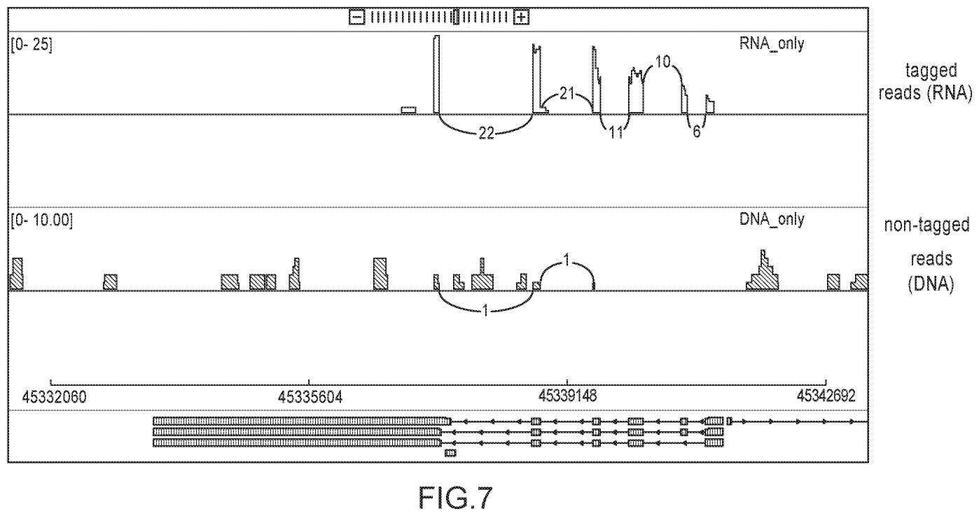

FIG. 7 provides another example of separated tagged and non-tagged reads showing expected mapping to genomic features. An exemplary locus for the different genome alignment of separated tagged and non-tagged libraries from index2 is shown. Sashimi plot showing spliced reads for TPT1 only in RNA and reads aligning with the position of annotated exons, without signal in intergenic or intronic regions (top signal). Only one read shows splicing in the DNA only data set (middle row), and the reads map to intergenic or intronic space. This is consistent with the tagged read-pairs originating from RNA and the non-tagged read-pairs originating from DNA. TPT1 (and FTH1 and RNR1 below) were selected as representative genes due to their expression.

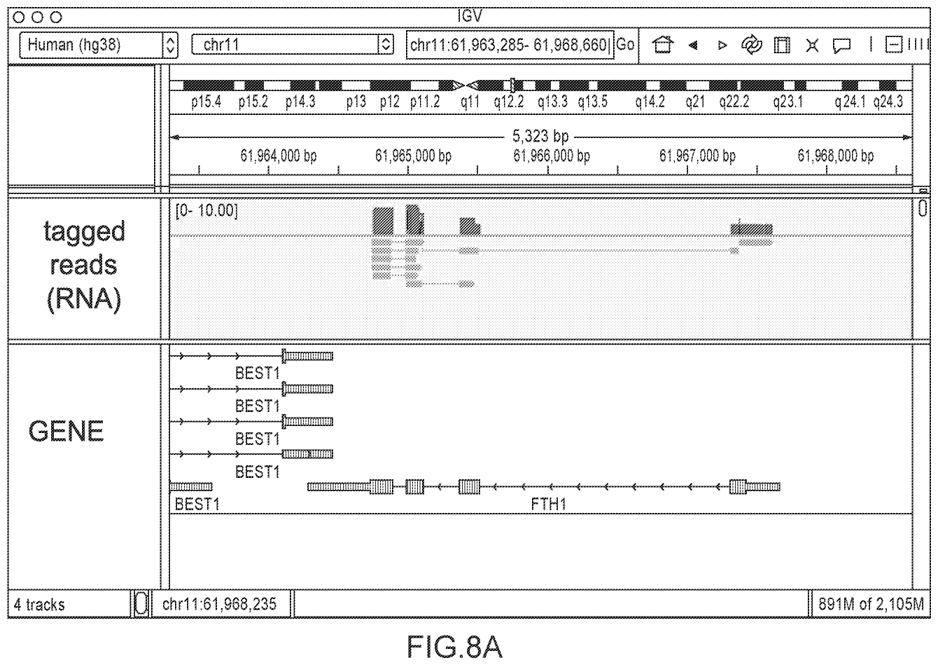

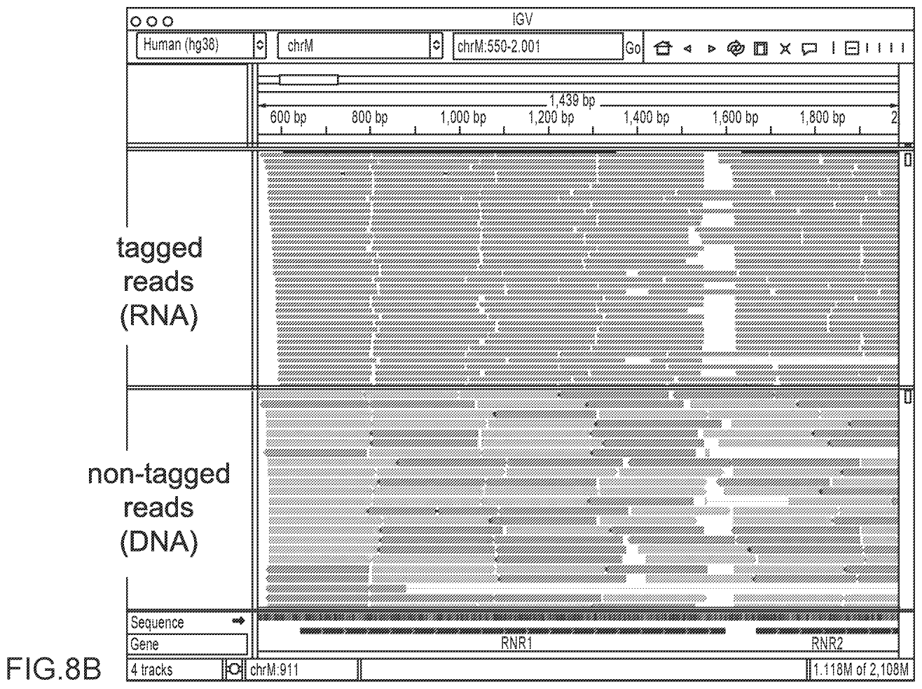

FIG. 8A-8B show the separated RNA library mapping to the expected strand of the annotated mRNA genes, demonstrating that reads which contain the tag sequence, expected to be corresponding to the first strand cDNA, map to the opposite strand of annotated mRNAs. The untagged DNA reads are not expected to show this stranded mapping. The bottom frame (“gene”) reads map to the minus (−) strand and the top frame reads map to the plus strand (+). In FIG. 8A, the annotated gene FTH1 is on the (−) strand of the genome, transcribing from left to right in this picture. The tagged reads align only to the reverse strand of the annotated gene, the (+) strand, as expected for a first strand cDNA (compare the graphic depiction in FIG. 1). In FIG. 8B, the annotated gene RNR1 is on the (+) strand of the genome, transcribing from right to left in this picture. The tagged reads (top frame) align only to the reverse strand of the annotated gene, the (−) strand, as expected for a first strand cDNA (compare the graphic depiction in FIG. 1). In comparison, reads from the non-tagged library (bottom frame) in FIG. 8B do not show a similar strandedness. In addition, the strandedness of all reads was estimated by calculating the fraction of the reads in the RNA reads with the correct orientation of the tag that mapped to the appropriate strand in the gene annotation. From all reads of index2 which contain the RNA tag, 98.8% of the reads map to the expected strand (reverse strand of annotated mRNA, the first strand cDNA). This is consistent with the tagged read-pairs originating from RNA and the not-tagged read-pairs originating from DNA.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following.

### REFERENCES

Abravaya, K., J. J. Carrino, S. Muldoon, and H. H. Lee. 1995 Detection of point mutations with a modified ligase chain reaction (Gap-LCR). Nucleic Acids Res. 23:675-82.
Al-Nedawi, K., B. Meehan, J. Micallef, V. Lhotak, L. May, A. Guha, and J. Rak. 2008. Intercellular transfer of the oncogenic receptor EGFRvIII by microvesicles derived from tumour cells. Nat Cell Biol. 10:619-24.
Balzar, M., M. J. Winter, C. J. de Boer, and S. V. Litvinov. 1999. The biology of the 17-1A antigen (Ep-CAM). J Mol Med. 77:699-712.
Cheruvanky, A., H. Zhou, T. Pisitkun, J. B. Kopp, M. A. Knepper, P. S. Yuen, and R. A. Star. 2007. Rapid isolation of urinary exosomal biomarkers using a nanomembrane ultrafiltration concentrator. Am J Physiol Renal Physiol. 292:F1657-61.
Cotton, R. G., N. R. Rodrigues, and R. D. Campbell. 1988. Reactivity of cytosine and thymine in single-base-pair mismatches with hydroxylamine and osmium tetroxide and its application to the study of mutations. Proc Natl Acad Sci USA. 85:4397-401.
Fischer, S. G., and L. S. Lerman 1979a. Length-independent separation of DNA restriction fragments in two-dimentional gel electrophoresis. Cell. 16:191-200.
Fischer, S. G., and L. S. Lerman 1979b. Two-dimensional electrophoretic separation of restriction enzyme fragments of DNA. Methods Enzymol. 68:183-91.
Guatelli, J. C., K. M. Whitfield, D. Y. Kwoh, K. J. Barringer, D. D. Richman, and T. R. Gingeras. 1990. Isothermal, in vitro amplification of nucleic acids by a multienzyme reaction modeled after retroviral replication. Proc Natl Acad Sci USA. 87:1874-8.
Hahn, P. J. 1993. Molecular biology of double-minute chromosomes. Bioessays. 15:477-84.
Kwoh, D. Y., G. R. Davis, K. M. Whitfield, H. L. Chappelle, L. J. DiMichele, and T. R. Gingeras. 1989. Transcription-based amplification system and detection of amplified human immunodeficiency virus type 1 with a bead-based sandwich hybridization format. Proc Natl Acad Sci USA. 86:1173-7.
Landegren, U., R. Kaiser, J. Sanders, and L. Hood. 1988. A ligase-mediated gene detection technique. Science. 241:1077-80.
Li, J., L. Wang, H. Mamon, M. H. Kulke, R. Berbeco, and G. M. Makrigiorgos. 2008. Replacing PCR with COLD-PCR enriches variant DNA sequences and redefines the sensitivity of genetic testing. Nat Med. 14:579-84.
Miele, E. A., D. R. Mills, and F. R. Kramer. 1983. Autocatalytic replication of a recombinant RNA. J Mol Biol. 171:281-95.
Myers, R. M., Z. Larin, and T. Maniatis. 1985. Detection of single base substitutions by ribonuclease cleavage at mismatches in RNA:DNA duplexes. Science. 230:1242-6.
Nagrath, S., L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, and M. Toner. 2007. Isolation of rare circulating tumour cells in cancer patients by microchip technology. Nature. 450:1235-9.
Nakazawa, H. D. English, P. L. randell, K. Nakazawa, N. Martel, B. K. Armstrong, and H. Yamasaki. 1994. UV and skin cancer: specific p53 gene mutation in normal skin as a biologically relevant exposure measurement. Proc Natl Acad Sci USA. 91:360-4.
Orita, M., H. Iwahana, H. Kanazawa, K. Hayashi, and T. Sekiya. 1989. Detection of polymorphisms of human DNA by gel electrophoresis as single-strand conformation polymorphisms. Proc Natl Acad Sci USA. 86:2766-70.
Raposo, G., H. W. Nijman, W. Stoorvogel, R. Liejendekker, C. V. Harding, C. J. Melief, and H. J. Geuze. 1996. B lymphocytes secrete antigen-presenting vesicles. J Exp Med. 183:1161-72.
Skog, J., T. Wurdinger, S. van Rijn, D. H. Meijer, L. Gainche, M. Sena-Esteves, W. T. Curry, Jr., B. S. Carter, A. M. Krichevsky, and X. O. Breakefield. 2008. Glioblastoma microvesicles transport RNA and proteins that promote tumour growth and provide diagnostic biomarkers. Nat Cell Biol. 10:1470-6.
Steemers, F. J., W. Chang, G. Lee, D. L. Barker, R. Shen, and K. L. Gunderson. 2006. Whole-genome genotyping with the single-base extension assay. Nat Methods. 3:31-3.
Taylor, D. D., and C. Gercel-Taylor. 2008. MicroRNA signatures of tumor-derived exosomes as diagnostic biomarkers of ovarian cancer. Gynecol Oncol. 110:13-21.
Went, P. T., A. Lugli, S. Meier, M. Bundi, M. Mirlacher, G. Sauter, and S. Dimhofer. 2004. Frequent EpCam protein expression in human carcinomas. Hum Pathol. 35:122-8.

What is claimed is:

1. A method for identifying a nucleic acid component in a mixture comprising:
a. providing a mixture comprising at least an RNA component and a DNA component;
b. specifically modifying the RNA component to provide a modified RNA component and an unmodified DNA component, or specifically modifying the DNA component to provide a modified DNA component and an unmodified RNA component; and
c. performing a molecular assay which identifies the modified RNA component or the modified DNA component.

2. The method of claim 1, wherein (a) comprises a mixture of exosomal RNA, exosomal DNA and cell-free DNA (cfDNA).

3. The method of claim 1, wherein (a) comprises a mixture of exosomal RNA and exosomal DNA.

4. The method of claim 1, wherein (a) comprises a mixture of exosomal RNA and cfDNA.

5. The method of claim 1, wherein (a) comprises a mixture of RNA and DNA from fresh, frozen or formalin fixed paraffin embedded (FFPE) tissue.

6. The method of any of the preceding claims, wherein (b) comprises chemically modifying the nucleic acid bases or backbone of the RNA component.

7. The method of any of the preceding claims, wherein (b) comprises modifying the nucleic acid sequence of the RNA component.

8. The method of any of the preceding claims, wherein (b) comprises adding a nucleic acid sequence to the RNA component.

9. The method of any of the preceding claims, wherein (b) comprises contacting the RNA component with one or more enzymes.

10. The method of any of the preceding claims, wherein (b) comprises contacting the RNA component with one or more reverse transcription enzymes.

11. The method of any of the preceding claims, wherein (b) comprises tagging the RNA component with a nucleic acid sequence.

12. The method of any of the preceding claims, wherein (b) comprises performing a first strand cDNA synthesis and contacting the RNA component with a reverse transcription enzyme and one or more tagged primers as a substrate for reverse transcription, thereby tagging the RNA component with a nucleic acid sequence.

13. The method of claim 12, wherein the one or more tagged primers comprise an oligonucleotide that comprises random nucleotides and nonrandom nucleotides.

14. The method of claim 12 or 13, wherein the one or more tagged primers comprise tagged random hexamers.

15. The method of claim 13, wherein the nonrandom nucleotide portion of the oligonucleotide comprises a G-C content in the range of about 40-60%, such as about 40-50%, or even about 40-45%.

16. The method of claim 13 or 15, wherein the nonrandom nucleotide portion of the oligonucleotide comprises a nucleic acid sequence that is of size the range of about 1-40, 1-20, or even 1-5 nucleotides.

17. The method of claim 13 or 15-16, wherein the nonrandom nucleotide portion of the oligonucleotide comprises a nucleic acid sequence that is unique to the genome of interest.

18. The method of claim 13 or 15-17, wherein the random nucleotide portion of the oligonucleotide is depleted of nucleotide combinations priming at highly abundant RNAs.

19. The method of any of the preceding claims, wherein (b) comprises modifying at least 50%, such as at least 60%, at least 70%, at least 80%, at least 85%, at least 90%, at least 95%, at least 98%, or even 100% of the RNA component in the mixture.

20. The method of any of the preceding claims, wherein (c) comprises detecting the modified RNA component by a polymerase chain reaction (PCR)-based assay.

21. The method of any of the preceding claims, wherein (c) comprises detecting the modified RNA component by next generation sequencing (NGS).

22. The method of any of the preceding claims, wherein (c) comprises detecting the modified RNA component and the DNA component by NGS in a single combined library.

23. The method of any of the preceding claims, wherein (c) comprises performing NGS under conditions sufficient to allow for efficient capture of both cDNA derived from the RNA component and the DNA component in a single combined library.

24. The method of any of the preceding claims, wherein (c) comprises distinguishing sequence reads from RNA and DNA in a NGS dataset.

25. The method of any of the preceding claims, wherein (c) comprises identifying the genomic strand of RNA origin.

26. The method of any of claims 1-5, wherein (b) comprises chemically modifying the nucleic acid bases or backbone of the DNA component

27. The method of claim 26, wherein (b) comprises modifying the nucleic acid sequence of the DNA component.

28. The method of any of claims 26-27, wherein (b) comprises adding a nucleic acid sequence to the DNA component.

29. The method of any of claims 26-28, wherein (b) comprises contacting the DNA component with one or more enzymes.

30. The method of any of claims 26-29, wherein (b) comprises tagging the DNA component with a nucleic acid sequence.

31. The method of claim 29, wherein (b) comprises tagging the DNA component with an oligonucleotide that comprises random nucleotides and nonrandom nucleotides.

32. The method of claim 31, wherein the nonrandom nucleotide portion of the oligonucleotide comprises a nucleic acid sequence that is unique to the genome of interest.

33. The method of any of claims 26-32, wherein (b) comprises modifying at least 50%, such as at least 60%, at least 70%, at least 80%, at least 85%, at least 90%, at least 95%, at least 98%, or even 100% of the DNA component in the mixture.

34. The method of any of claims 26-33, wherein (c) comprises detecting the modified DNA component by a polymerase chain reaction (PCR)-based assay.

35. The method of any of claims 26-34, wherein (c) comprises detecting the modified DNA component by next generation sequencing (NGS).

36. The method of any of claims 26-35, wherein (c) comprises detecting the modified DNA component and the RNA component by NGS in a single combined library.

37. The method of any of claims 26-36, wherein (c) comprises identifying the genomic strand of DNA origin.

38. The method of any of claims 1-37, further comprising performing a transcriptome analysis and detecting genomic or epigenomic features of the genome.

39. The method of any of claims 1-38, further comprising determining relative abundance of the RNA component and the DNA component in order to identify copy number variations present in the mixture.

40. The method of any of claims 1-39, further comprising predicting expressed, relevant tumor Neoantigens.

41. The method of any of claims 1-40, further comprising simultaneous mutation detection in the RNA component and the DNA component of the mixture.
